(19) United States
(12) Patent Application Publication
(10) Pub. No.: US 2005/0215998 A1
(43) Pub. Date: Sep. 29, 2005

(54) Device for joining a longitudinal support with a bone fixation means

(76) Inventor: Raoul Donath, Grenzach-Wyhlen (DE)

Correspondence Address: JONES DAY, 222 EAST 41ST STREET, NEW YORK, NY, 10017-6702, US

(21) Appl. No.: 10/509,075

(22) PCT Filed: Nov. 22, 2001
(86) PCT No.: PCT/CH01/00682

§ 371 Date: Sep. 22, 2004

(51) Int. Cl. A61B 17/56

(52) U.S. Cl. 606/61

(57) ABSTRACT

The present invention is directed to a device for connecting a longitudinal carrier, for example, a longitudinal spinal rod, to a bone fixation means, for example, a pedicle screw. The device includes a connection element having an upper end, a lower end, and a cavity extending from the upper end to the lower end, the cavity having a reduced diameter portion at the lower end thereof thereby forming a shoulder for engaging the head of the bone fixation means. The connection element further including a transverse channel for receiving the longitudinal carrier. The device further includes a sealing cap incorporating tensioning means for securing the longitudinal carrier within the transverse channel. The sealing cap being secured to the connection element by way of arresting means, wherein the arresting means preferably includes a plurality of bulges, i.e., projections, formed on the outer surface of the connection element and the sealing cap includes a plurality of complementary depressions, i.e., recesses. The device may further incorporate securing means in order to prevent the bone fixation means from passing through the upper end of the cavity once the bone fixation means has been introduced into the connection element.

### BACKGROUND OF THE INVENTION

The invention relates to a device for connecting a longitudinal carrier, for example, a longitudinal spinal rod, to a bone fixation means, for example, a pedicle screw.

Devices are already known in the art for connecting pedicle screws to longitudinal carriers for fixing the vertebral column. For example, U.S. Pat. No. 5,584,834 to Errico discloses a device for connecting a bone fixation element to a longitudinal carrier.

The connection device generally consists of a cylindrical central body, which is provided at its lower end with a slotted, tapered outer surface and a hollow-spherical cavity for receiving a spherical head of a bone fixation element and an externally threaded upper end. In its central section, the central body includes a channel, opened on the side, running perpendicular to the central axis for receiving the longitudinal carrier. The connection device further includes a locking collar and a rod securing sleeve. The locking collar has an inner tapered surface corresponding to the outer tapered surface formed on the lower end of the central body while the rod securing sleeve has a passage opening towards the locking collar. When in the assembled state, the locking collar and the rod securing sleeve are axially displaceable with respect to the central body by means of a nut, which can be screwed over the external thread formed on the upper end of the central body. In use, the longitudinal carrier is inserted between the locking collar and the rod securing sleeve so that the longitudinal carrier passes through the channel formed in the central body and the passage formed in the rod securing sleeve. Thereafter, rotation of the nut causes the rod securing sleeve to move downwards, which presses the longitudinal carrier inserted in the channel onto the locking collar, which, in turn, causes the inner tapered surface of the locking collar to pass over the external tapered surface of the central body. As a result, the lower end of the central body compresses, which causes the position of the spherical head of the bone fixation element which is received within the hollow-spherical cavity of the central body to be fixed.

The disadvantage of this prior art device is, on the one hand, the significant construction height of the connection element due in part to the ball and socket type connection between the spherical head of the bone fixation element and the central body and, on the other hand, the space required for the surgical instrument for tightening the nut over the external thread formed on the upper end of the central body.

### SUMMARY OF THE INVENTION

The invention is intended to remedy this situation. The invention discloses a device that requires as little space as possible and which can be implanted with a reduced number of surgical instruments and steps. Specifically, it would be preferable, to insert the implant with a surgical instrument which has a diameter no larger than the diameter of the implant so that as little damage as possible is caused to the surrounding tissue parts.

The invention discloses a device for connecting a longitudinal carrier to a bone fixation means, specifically a pedicle screw, the device includes a connection element having a central axis, an external surface, an upper end, a lower end, a cavity extending coaxially along the central axis from the upper end to the lower end, the cavity having a reduced diameter portion at the lower end thereof, thereby forming at least one shoulder therein. The connection element further including a transverse channel passing through the connection element for receiving the longitudinal carrier. The device also includes a sealing cap having a front end, a rear end, a second cavity opening at the front end for receiving the connection element, and a second channel extending transversely to the central axis opening towards the front end for receiving the longitudinal carrier inserted within the transverse channel. The sealing cap may also incorporate tensioning means for fixedly securing the position of the longitudinal carrier. The device is characterized in that the connection element and the sealing cap contain complementary arresting means for securing the sealing cap to the connection element.

In a further embodiment of the device according to the invention, the device comprises a connection element with a sealing cap and a tensioning means. The connection element has a central axis, an upper end, a lower end, and a cavity extending coaxially along the central axis from the upper end to the lower end of the connection element. The cavity has a reduced diameter section at the lower end of the connection element which forms a shoulder. The connection element further includes a transverse channel that is open at the upper end of the connection element so that a longitudinal carrier, for example, a longitudinal carrier representing a vertebral fixation system can be received within the channel, substantially orthogonal to the central axis, whereas a bone fixation means, for example, a pedicle screw can be introduced through the cavity parallel to the central axis, until the screw head of the pedicle screw rests on the shoulder formed in the cavity. The sealing cap is provided with a second channel, which is also arranged transverse to the central axis and which is opened towards the front end of the sealing cap so that, the longitudinal carrier may be received by the sealing cap when the sealing cap is installed on the connection element. The second channel divides the sealing cap into two segments, which are elastically deformable with respect to the central axis so that, in use, the sealing cap may be fixed with respect to the connection element by arresting means. The rear end of the sealing cap may further include tensioning means, which, when tightened, fix the position of the longitudinal carrier and the bone fixation means with respect to the connection element.

In the preferred embodiment of the device according to the invention, the arresting means are arranged on the periphery of the connection element and also on the periphery of the cavity in the sealing cap. The arresting means preferably includes a plurality of bulges formed on the connection element and a plurality of complementary depressions formed in the cavity of the sealing cap.

In another embodiment of the device according to the invention, the shoulder that narrows the cavity at the lower end of the sealing cap comprises a planar bearing surface for receiving and supporting, for example, the screw head of a pedicle screw. Alternatively, the bearing surface can also be designed in spherical form or having several concentric steps.

The present invention provides a device with a very low structural height. Moreover, the present invention provides a device which can maintain the longitudinal carrier within the transverse channel formed in the connection element by means of a single locking mechanism. The present invention also provides a device which can facilitate installation by way of an instrument which has a diameter less than the diameter of the implant, thus minimizing the size of the incision needed for accessing the operational site, which, in turn, reduces the amount of patient trauma and making the device ideal for minimum invasive or navigated surgery. These implants also offer a possibility of treating patients in cases of thorascopic access.

In a further embodiment the device according to the invention may include securing means which narrows the cavity formed in the connection element between the rear end of the head of the bone fixation means and the upper end of the connection element so that the bone fixation means can not pass through the upper end of the connection element. This enables the implants to be pre-assembled thus reducing the amount of time needed in the operating room and also reducing the risk for example, of mixing up or incorrectly inserting the bone fixation means into the connection elements.

The invention and further developments of the invention are described in more detail below on the basis of partially schematic illustrations of several embodiments.

### BRIEF DESCRIPTION OF THE DRAWINGS

FIG. 1 shows a longitudinal section through the preferred embodiment of the device according to the invention;

FIG. 2a shows a view of the connection element of the embodiment of the device according to the invention illustrated in FIG. 1;

FIG. 2b shows a view of the embodiment of the device according to the invention illustrated in FIG. 1;

FIG. 3 shows a longitudinal section through another embodiment of the device according to the invention;

FIG. 4 shows a view of the embodiment of the device according to the invention illustrated in FIG. 3;

FIG. 5 shows a longitudinal section through a further embodiment of the device according to the invention; and

FIG. 6 shows a longitudinal section through a further embodiment of the device according to the invention.

### DESCRIPTION OF THE PREFERRED EMBODIMENTS

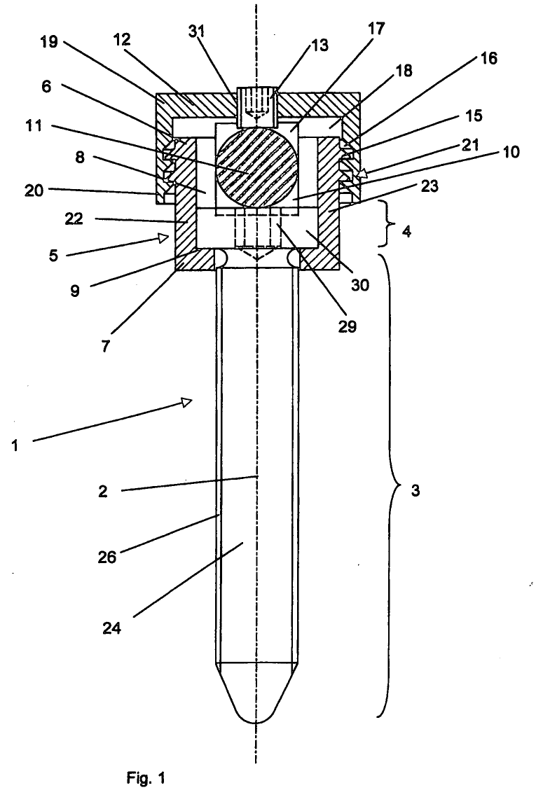

FIG. 1 illustrates a bone fixation means 1 designed as a pedicle screw together with a connection element 5, a longitudinal carrier 11, a sealing cap 12 axially displaceable over the upper end 6 of the connection element 5 and a tensioning means 13, i.e., a set screw, incorporated into the sealing cap 12. The bone fixation means 1 includes a front segment 3 and a rear segment 4, as shown, the front segment 3 has a screw shaft 24 with external thread 26 so that the bone fixation means 1 may threadedly engage a patient's pedicle, while the rear segment 4 has a circular-cylindrical screw head 30 having means 29, illustrated as a hexagon socket, for receiving a screwdriver, which are arranged at an end on the screw head 30. Alternatively, the means 29 for receiving a screwdriver may be in the form, for example, Torx or Phillips.

As shown, the connection element 5 generally is in the form of a hollow body having a central axis 2, an upper end 6, a lower end 7, and a cavity 8. The diameter of the cavity 8 formed in the connection element 5 is configured so as to have a reduced diameter portion at the lower end 7 of the connection element 5, thus forming a shoulder 9 with a bearing surface 25, preferably a planar bearing surface, on which the screw head 30 of the bone fixation means 1 can be placed. The lower end 7 of the connection element 5 is further configured so that the screw shaft 24 of the bone fixation means 1 can be passed through the reduced diameter portion of the connection element 5. The connection element 5 further includes a first channel 10, generally extending transverse to the central axis 2, wherein the first channel 10 is open towards the upper end 6 of the connection element 5 so that the longitudinal carrier 11 may be inserted into the first channel 10 from the top. The depth of the channel 10 being sized and configured to receive the longitudinal carrier 11. More specifically, the depth of the channel 10, measured from the upper end 6 of the connection element 5 parallel to the central axis 2, is designed in such a way that a longitudinal carrier 11 is inserted into the channel 10 above the screw head 30 of the bone fixation means 1.

The sealing cap 12 is sized and configured so that, in use, the sealing cap 12 is axially displaceable, i.e., pushed, over the connection element 5 so that the front end 20 of the sealing cap 12 is directed towards the lower end 7 of the connection element 5 and the connection element 5 is partially received parallel to the central axis 2 in a second cavity 18 formed in the sealing cap 12. The sealing cap 12 also incorporates a second channel 17 which enables the acceptance of the longitudinal carrier 11 inserted in the connection element 5 in the first channel 10 to be received in the sealing cap 12. The second channel 17 is open at the front end 20 of the sealing cap 12 so that, in use, when the sealing cap 12 is axially displaced over the connection element 5, the second channel 17 receives the longitudinal carrier 11 inserted in the connection element 5.

The device may also include arresting means 21 for securing the connection element 5 and the sealing cap 12. More specifically, the outer surface of the connection element 5 may include bulges 15 while the inner surface of the sealing cap 12, and more specifically, the inner surface of the second cavity 18, includes complementary depressions 16 for engaging the bulges 15. Preferably, the connection element 5 and the sealing cap 12 include a plurality of arresting means 21 so that different latch positions are possible. That is, preferably, the arresting means 21 includes a plurality of bulges 15 and a plurality of depressions 16 so that when the first depression 16 has been pushed over the first bulge, i.e., the first latch position, the longitudinal carrier 11 may still be longitudinally moved with respect to the bone fixation means 1 so that the bones and/or bone fragments affixed thereto may be repositioned. However, as the sealing cap 12 is further axially displaced over the connection element 5 such that subsequent bulges 15 are aligned with subsequent depressions 16, the longitudinal carrier 11 becomes fixed with respect to the bone fixation means 1.

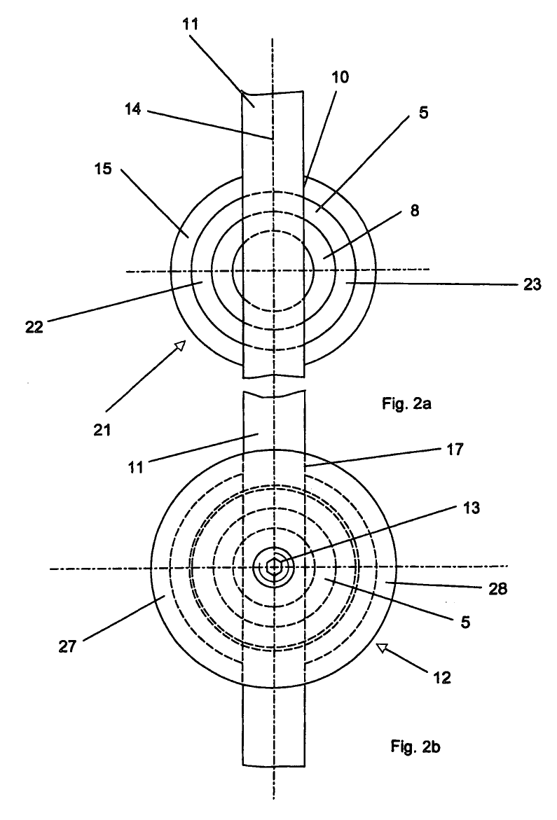

Preferably, as shown, the bulges 15 and the depressions 16 are provided with a saw-tooth shaped profile, when viewed in a cross section surface parallel to the central axis 2, so that the steep flanks of the bulges 15 are oriented towards the lower end 7 of the connection element 5 and the steep flanks of the depressions 16 are oriented towards the rear end 19 of the sealing cap 12 such that, in use, as the sealing cap 12 is axially displaced over the connection element 5 parallel to the central axis 2, the sealing cap 12, and more specifically, the two segments 27, 28 (FIG. 2b) of the sealing cap 12 formed by the second channel 17 are more easily biased apart, i.e., resiliently spread transverse to the central axis 2. This enables the sealing cap 12 to pass over the bulges 15 formed on the connection element 5 until the bulges 15 align with the depressions 16 formed in the second cavity 18, at which point the two segments 27, 28 (FIG. 2b) resiliently move back, i.e., elastically move towards the central axis 2, so that the bulges 15 engage the depressions 16.

The sealing cap 12 may further incorporate tensioning means 13, which as shown is in the form of a set screw that can be screwed into an internally threaded hole 31 formed at the rear end 19 of the sealing cap 12 coaxial to the central axis 2. Rotation of the tensioning means 13 causes the tensioning means 13 to press against the longitudinal carrier 11, which in turn causes the longitudinal carrier 11 to press against the head 30 of the bone fixation means 1 resulting in the head 30 being clamped between the shoulder 9 formed at the lower end 7 of the connection element 5 and the longitudinal carrier 11 thereby fixing the position of the bone fixation means 1 together with the longitudinal carrier 11 with respect to the connection element 5.

FIG. 2 shows the connection element 5 from the perspective of the upper end 6. As shown, the cavity 8 and the arresting means 21 are arranged concentrically about the central axis 2 (FIG. 1), wherein the arresting means 21 is configured in the form of bulges 15 that are, when seen parallel to the central axis 2, arranged on the outer circumference of the connection means 5. The ring-shaped side wall of the connection element 5 and the bulges 15 are interrupted by the channel 10, wherein the channel axis 14 runs transverse to the central axis 2 (FIG. 1).

The longitudinal carrier 11 is inserted in the channel 10. In addition, the channel 10 forms, on the connection element 5, two sidewalls 22, 23 for a section of its length, wherein the bulges 15 are provided only on these two sidewalls 22, 23 and do not surround the entire connection element 5.

FIG. 2b shows the sealing cap 12 with the tensioning means 13 assembled onto the connection element 5. As shown, the second channel 17 formed in the sealing cap 12 forms two segments 27, 28 in the sealing cap 12, which resiliently spread radially when the sealing cap 12 is axially displaced. i.e., pushed, over the connection element 5, which allows the sealing cap 12 to be pushed over the bulges 15 formed on the connection element 5 (FIG. 1). As soon as the sealing cap 12 is displaced far enough over the connection element 5 so that the depressions 16 (FIG. 1) in the sealing cap 12 align with the bulges 15 formed on the connection element 5 (FIG. 1), the two segments 27, 28 will elastically snap back towards the central axis 2 (FIG. 1), wherein the bulges 15 formed on the connection element 5 will engage the depressions 16 formed on the sealing cap 12.

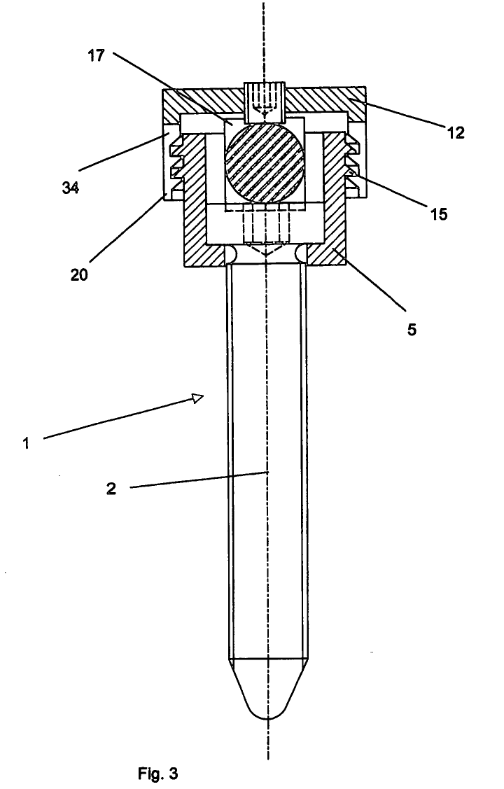

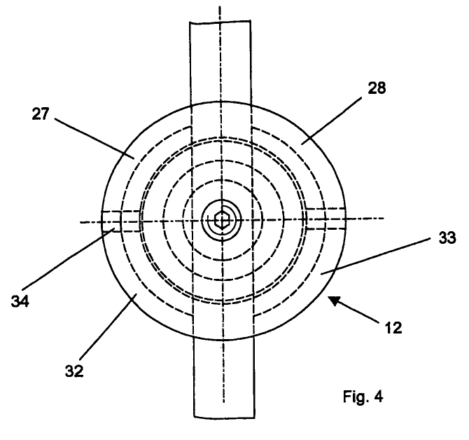

The embodiment of the device according to the invention illustrated in FIG. 3 and FIG. 4 differs from the embodiment illustrated in FIGS. 1 and 2 only in that the sealing cap 12 is provided with two slots 34 orthogonal to the second channel 17, which are formed in the wall of the sealing cap 12, the slots 34 extending from the front end 20 of the sealing cap 12. The slots 34 divide the sealing cap 12 into four segments 27, 28, 32, 33 thereby increasing the elasticity of the sealing cap 12 so that the sealing cap 12 may be more easily axially displaced over the bulges 15 formed on the connection element 5 as compared to a sealing cap 12 having only two segments 27, 28 as described above.

In use, the pre-assembled implant comprising the bone fixation means 1 and the connection element 5 may be removed from an implant carrying container by a screw driver which engages the means 29, without any need for a further instrument for holding the implant and without a surgeon having to join the bone fixation means 1 and the connection element 5 together. Thereafter, the pre-assembled implant may be screwed into the prepared pedicle. After the longitudinal carrier 11 is inserted into the first channel 10 arranged in the connection element 5, the same screwdriver may again be used to remove the pre-assembled sealing cap 12 provided with the tensioning means 13 from the implant carrying container, in which operation there is again no need for a special holding instrument. By means of a special clamp, the sealing cap 12 is locked into the first latch position, i.e., the first depression 16 formed in the sealing cap 12 is pushed over and aligned with the first bulge 15 formed on the connection element 5 so that the longitudinal carrier 11 inserted within the first channel 10 is prevented from escaping from the channel 10 formed in the connection element 5 but the longitudinal carrier 11 is free to longitudinally move with respect to the connection element 5 so that the bone and/or bone fragments attached to the bone fixation means 1 may be repositioned. After repositioning is completed, the sealing cap 12 is placed in the second or third latch position and locked. The tensioning means 13 is then tightened by means of the screwdriver so that the longitudinal carrier 11 inserted in the first channel 10 is pressed onto the screw head 30 of the bone fixation means 1, which, in turn, clamps the head 30 in-between the shoulder 9 formed on the lower end 7 of the connection element 5 and the longitudinal carrier 11, thus fixing the position of the bone fixation means 1 together with the longitudinal carrier 11 with respect to the connection element 5.

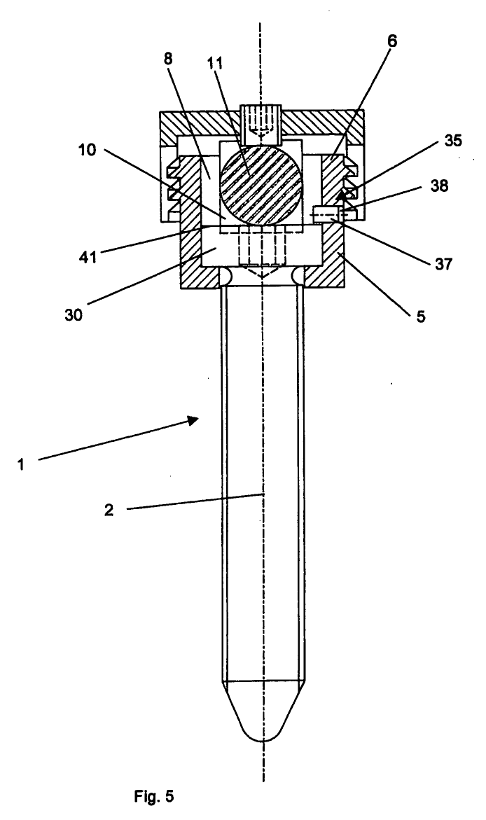

FIG. 5 illustrates an embodiment of the device according to the invention that differs from the embodiments described above only in that the screw head 30 of the bone fixation means 1 is secured against falling out of the cavity 8 by means of a securing means 35. As shown, the securing means 35 comprises a pin 37 that is pressed, for example, in a hole 38 running transverse to the central axis 2 and which extends into the cavity 8. The hole 38 is arranged axially between the rear end 41 of the screw head 30 of the bone fixation means 1 and the upper end 6 of the connection element 5. Alternatively, the connection element 5 may include several holes 38 spread across the circumference of the connection element 5 for receiving several pins 37.

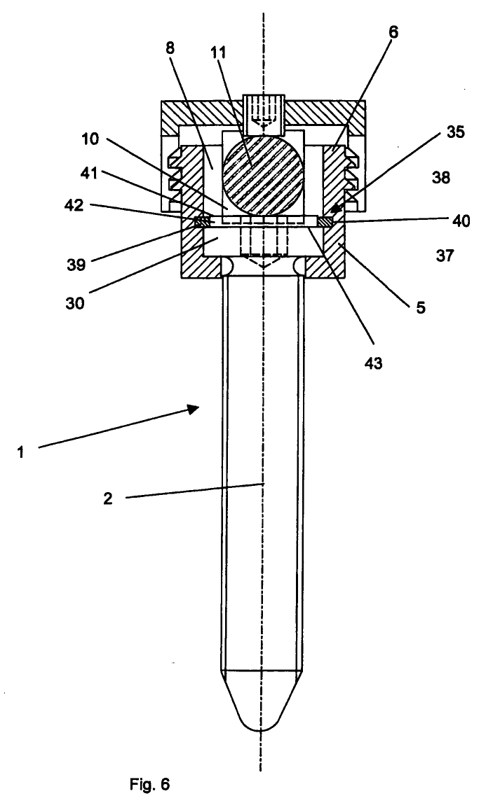

FIG. 6 shows an embodiment of the device according to the invention wherein the securing means 35 may be in the form of a snap ring 39. The snap ring 39 is received within a groove 40 formed on the rear end 41 of the screw head 30 and which protrudes into the cavity 8. The groove 40 is arranged axially in such a way that the snap ring 39 inserted therein does not extend beyond the rear end 41 of the screw head 30 towards the upper end 6 of the connection element 5, which means that the longitudinal carrier 11 does not lie on the snap ring 39. To achieve this, for example, the screw head 30 is designed with a reduced diameter portion at its rear end 41. That is, the screw head 30 is provided at this rear end 41 with an axial segment 42 which has a diameter smaller than the screw head 30 thus forming a shoulder 43 for receiving the securing means 35 provided here in the form of a snap ring 39. The reduced diameter portion 42 of the screw head 30 passes through the ring-shaped opening of the snap ring 39 and protrudes beyond the snap ring 39, so that the longitudinal carrier 11 can rest on the rear end 41 of the screw head 30. Therein, in use, the securing means 35 prevents the screw head 30 from axial movement towards the upper end 6 of the connection element 5.

1. A device for connecting a longitudinal carrier to a bone fixation means, the device comprising
a connection element having a central axis, an external surface, an upper end, a lower end, a cavity extending coaxially along the central axis from the upper end to the lower end, the cavity having a reduced diameter portion at the lower end forming at least one shoulder therein, and a channel passing through the connection element transversely to the central axis for receiving the longitudinal carrier;
a sealing cap having a front end, a rear end, a second cavity opening at the front end for receiving the connection element, and a second channel extending transversely to the central axis and opening towards the front end of the sealing cap; and
tensioning means for engaging the rear end of the sealing cap for securing the position of the longitudinal carrier inserted in the channel with respect to the connection element
wherein the external surface of the connection element and the internal surface of the second cavity formed in the sealing cap contains complementary arresting means for securing the sealing cap to the connection element, and
wherein the device further includes securing means so that the bone fixation means is prevented from passing through the cavity.

2. The device according to claim 1, wherein the arresting means are arranged orthogonal to the central axis on the periphery of the connection element and on the periphery of the second cavity in the sealing cap.

3. The device according to claim 2, wherein the arresting means includes a plurality of bulges formed on the external surface of the connection element and a plurality of complementary depressions formed in the second cavity of the sealing cap.

4. The device according to claim 1 wherein the shoulder has a level bearing surface of circular-ring shape.

5. The device according to claim 1 wherein the sealing cap further includes two slots arranged orthogonal to the second channel, the slots extending from the front end of the sealing cap.

6. The device according to claim 1, further comprising bone fixation means having a central axis, a front segment and an axially adjoining rear segment, wherein the rear segment has a cylindrical form for engaging the shoulder, and the front segment extends through the lower end of the connection element for engaging a bone.

7. The device according to claim 6, wherein the bone fixation means is a pedicle screw with a screw shaft having an external thread and a screw head at an end thereof.
